(19) United States
(12) Patent Application Publication
(10) Pub. No.: US 2007/0244486 A1
(43) Pub. Date: Oct. 18, 2007

(54) Reamer Guide and Method

(76) Inventors: Alex Hogg, Leeds (GB); Mick Rock, Leeds (GB); Kevin Booth, Leeds (GB)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 10/584,357

(22) PCT Filed: Dec. 21, 2004
(86) PCT No.: PCT/GB04/05341

§ 371 Date: Jun. 26, 2007

(30) Foreign Application Priority Data
Dec. 24, 2003 (GB) 0329870.0

(51) Int. Cl. A61B 17/17

(52) U.S. Cl. 606/87

(57) ABSTRACT

A reamer guide is described for reaming a cavity within a tibia. The reamer guide comprises: a plate having a circular aperture there through and defining a plane; and a disc rotatably mounted in the aperture, the disc having a bushing therein defining a reamer axis offset from the center of the disc for receiving a reamer in use and in which the disc is rotatable about an axis transverse to the plane of the plate. Thus a cavity larger than the reamer can be accurately and easily formed.

The present invention relates to reaming and in particular to a reaming guide and method for use in reaming a cavity in a bone, and in particular the tibia.

There is a general move toward minimally invasive surgical procedures. Minimally invasive surgery has a number of considerations, including the size of incisions used, which tends to have cosmetic consequences, and also the trauma suffered by body parts, such as soft tissues, ligaments, muscles, tendons and similar, affected by the surgical procedure at the surgical site.

During knee surgery, for example total knee replacement surgery, it can be required to attach a prosthesis to the proximal end of the tibia where it interfaces with the knee. In order to be mounted securely on the tibia, the prosthesis can include a stem which is located within a cavity formed within the proximal part of the tibia. Therefore as part of this procedure, it is necessary to drill or ream a cavity of the correct size and depth in the tibia to receive the stem of the prosthesis.

In one surgical procedure, a top surface of the tibia resected to a flat surface and then the femur is subluxed to a level sufficient to expose the resected flat top surface of the tibia. A plate is mounted on the surface and a large tower is mounted on the plate and extends substantially perpendicularly from the surface to receive a reamer.

In order to carry out such a known method of forming a cavity a large incision is required because the reamer must enter the guide along the axis of the cavity. This also requires the femur to be subluxed to a level sufficient to allow access of the reamer along the axis of the tibia which can result in damage to the tendons and other soft body parts adjacent the knee.

Hence the above method and reaming apparatus are not suitable for use in a minimally invasive procedure as large incisions are required and soft body parts at the surgical site can be damaged. Therefore, the present invention addresses problems associated with reaming a cavity as part of a minimally invasive surgical technique.

According to a first aspect of the present invention, there is provided a reamer guide for reaming a cavity within a bone, the reamer guide comprising:
a plate having a circular aperture there through and defining a plane; and
a disc rotatably mounted in the aperture, the disc having a bushing therein defining a reamer axis offset from the centre of the disc for receiving a reamer in use and in which the disc is rotatable about an axis transverse to the plane of the plate.

The present invention includes a reamer guide with a rotating disc located above where the cavity is required to be formed. The disc has an offset bushing for receiving a reamer. A larger diameter cavity than the diameter of the reamer can therefore be formed by rotating the disc (and therefore the reamer within the bushing). This allows a cavity to be formed with a relatively small reamer.

Such a reamer can therefore be used in a surgical procedure in which access to a surgical site, such as the top surface of the tibia is limited. Also, in the example of knee replacement surgery, the femur does not need to be subluxed to a level sufficient to carry out reaming with conventional reamer assemblies as described above, and so trauma, damage or disturbance of soft body parts, such as the quad extensor mechanism, is reduced or eliminated.

Although the terms disc and plate have been used to refer to the components of the reamer guide, it will appreciated that these components need not be perfectly planar. For example the disc and the plate could incorporate reinforcement or other projections on their surfaces. The term bushing is used to refer to an aperture for receiving and guiding the end of a reamer which is used to form the cavity.

Such a reamer guide allows a relatively small reamer to ream a cavity larger than the reamer by reaming while rotating the disc through 360 degrees. Thus, a cavity can be reamed with a smaller incision. Hence, as mentioned above, the femur does not have to be subluxed as far, reducing tension in the ligaments during surgery.

The plate can further include at least one mounting hole. In other embodiments, the plate can include at least two mounting holes for receiving fixings to secure the plate to the surface. The mounting holes can be located on opposed sides of the aperture. This allows the plate to be secured on the resected surface to ensure that it remains in the correct position to allow the cavity to be reamed accurately.

The plate can have an inner circular edge having a first formation, and the periphery of the disc can have a matching formation which engages with the first formation to retain the disc within the aperture. The circular first formation simplifies the use and assembly of the reamer guide and helps to prevent the disc from escaping from the aperture in the plate in use while still allowing the disc to rotate. The inner circular edge need not extend around the entire circumference of a circle as the aperture need not be wholly enclosed by the plate, depending on the size of the plate, and can have an open portion, similar to a ‘c’ shape.

The first formation can be a shoulder and the matching formation can be a flange. This allows the disc to be retained in the aperture with a simple construction that does not restrict the rotation of the disc within the aperture. It also allows the disc and plate to be disassembled easily for cleaning. In another embodiment, the first formation and matching formation can between them provide a tongue and groove.

The reamer axis can be angled toward the axis of rotation of the disc. This causes a conical-shaped cavity to result when the disc is rotated through 360 degrees together with a reamer.

The bushing can include a stop toward a free end for limiting the travel of a reamer into the bushing. This provides a safety feature against accidentally reaming a cavity which is too deep.

According to a further aspect of the invention, there is provided an assembly including:
a reamer guide according to the first aspect of the invention; and
a reamer sized to substantially match the inner diameter of the bushing.

The guide can therefore be used with a reamer by inserting the reaming end of the reamer into the bushing. Since the reamer has at least a portion which is sized to match the inner diameter of the bushing play and chatter between the reamer and bushing is reduced, allowing a more accurate cavity to be formed.

In the assembly, the reamer can further include a projection sized to abut against or engage with an edge of a free end of the bushing, the projection positioned a distance from a distal end of the reamer to control the depth of the cavity to be formed to correspond to a desired depth.

Thus the depth of cavity can be controlled to ensure it is correct for the length of the stem of a prosthesis to be installed in the cavity. The projection can be formed at a fixed position on the reamer, or can be movable to a variety positions. This allows the assembly to be adjusted to different cavity depths by changing the reamer, or adjusting the position of the stop, respectively. It will be appreciated that the position of the projection from the distal end of the reamer in most cases will not equal the desired depth; the position of the projection must include the depth of the bushing and disc along the axis of the reamer, and also take into account the angle of inclination of the reamer (if any).

The assembly can further comprise a universal joint attached to a proximal end of the reamer for transmitting torque to the reamer about the axis between the distal and proximal ends. Thus, torque can be provided to the reamer without requiring the drive shaft to extend along the axis of the reamer. This allows the reamer to be rotated easily through a small incision.

The assembly can further comprise a drive mechanism attached to the universal joint for rotating the reamer.

According to a further aspect of the invention, there is provided a method of reaming a cavity within a bone, the method comprising:
resecting a surface of the bone in which the cavity is to be reamed;
locating a reamer guide on the resected surface, the reamer guide having a rotating disc with a bushing offset from the centre of rotation of the disc, such that the centre of the disc is located above the desired centre of the cavity;
attaching a drive mechanism to a reamer, the drive mechanism extending at least partially at an acute angle to the longitudinal axis of the reamer;
reaming the bone through the bushing with the reamer; and
rotating the disc while still driving the reamer, thereby enlarging the cavity.

The method is particularly suitable for use on a tibia. It is therefore possible to ream a cavity in the bone, or tibia, which has a greater diameter than the reamer itself. This allows the use of a smaller reamer and consequently a smaller incision can be used. Further, when used on a tibia, this reduces the amount of subluxation of the femur required, which helps to obviate or reduce damage to any nearby soft body parts or structures.

Reaming through the bushing can include reaming to a predetermined depth before rotating the disc.

The method can further comprise securing the reamer guide on the resected surface before beginning reaming. This allows the cavity to be formed accurately with a reduced risk that the reamer guide will move during reaming.

An embodiment of the invention will now be described, by way example only and not in any limitative sense, with reference to the following drawings in which:

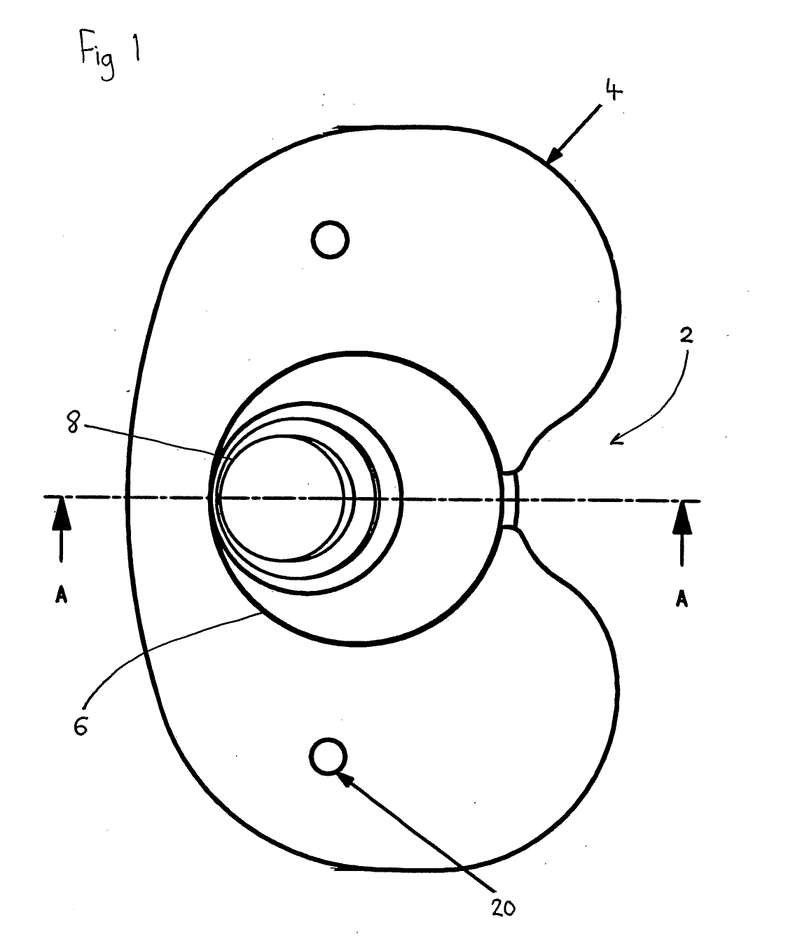

FIG. 1 shows a plan view of a reamer guide according to an embodiment of the present invention;

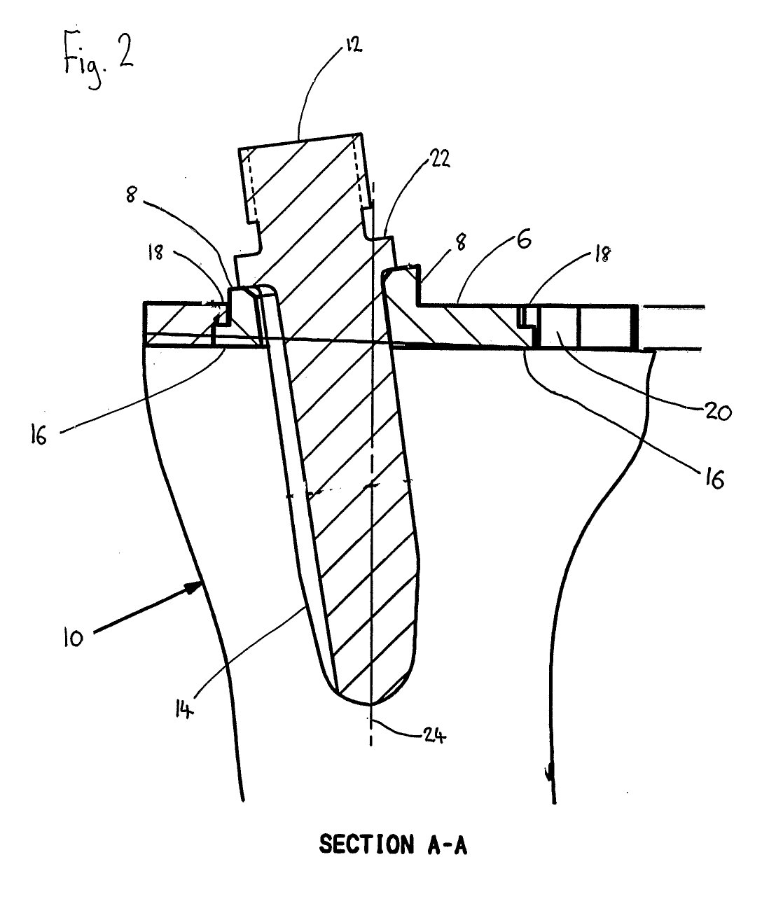

FIG. 2 is a cross section of the reamer guide of FIG. 1 installed on a tibia showing an operative position of a reamer;

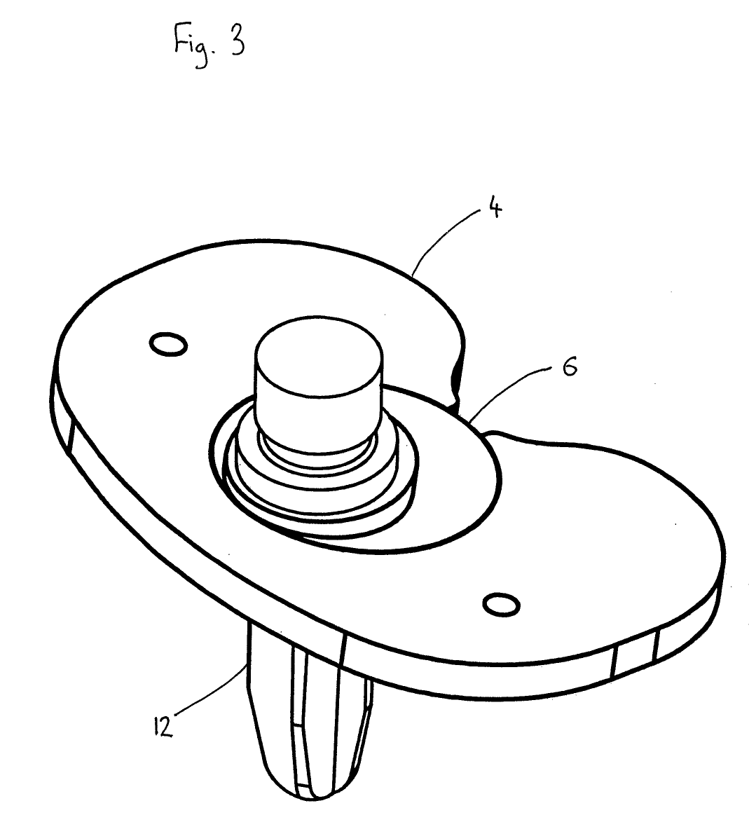

FIG. 3 is an isometric view of a reamer guide and reamer assembly according to an embodiment of the present invention and including the reamer guide shown in FIGS. 1 and 2;

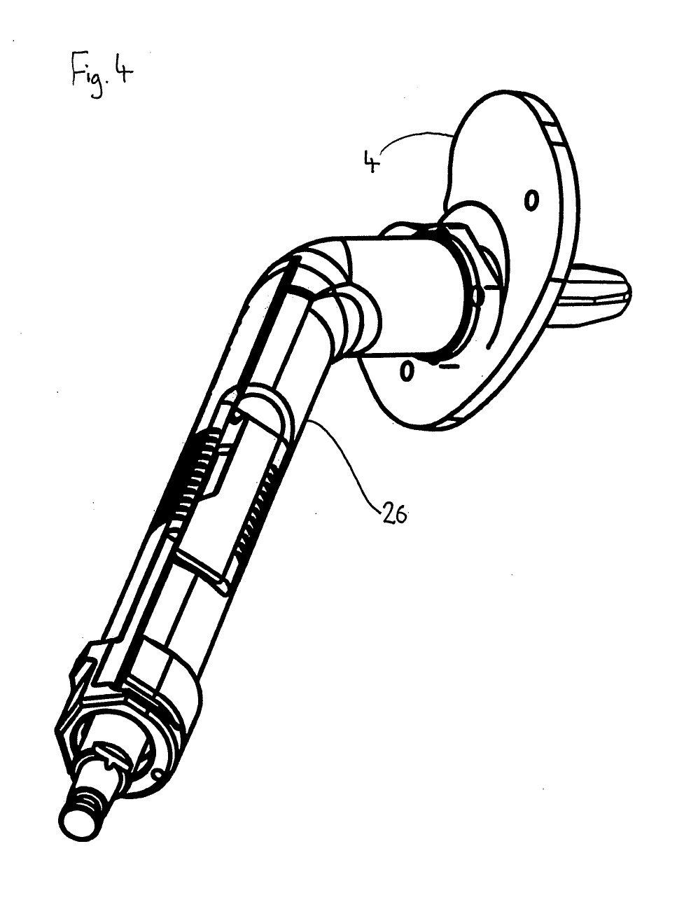

FIG. 4 is an isometric view of a reamer guide, reamer, and driver assembly according to an embodiment of the present invention; and

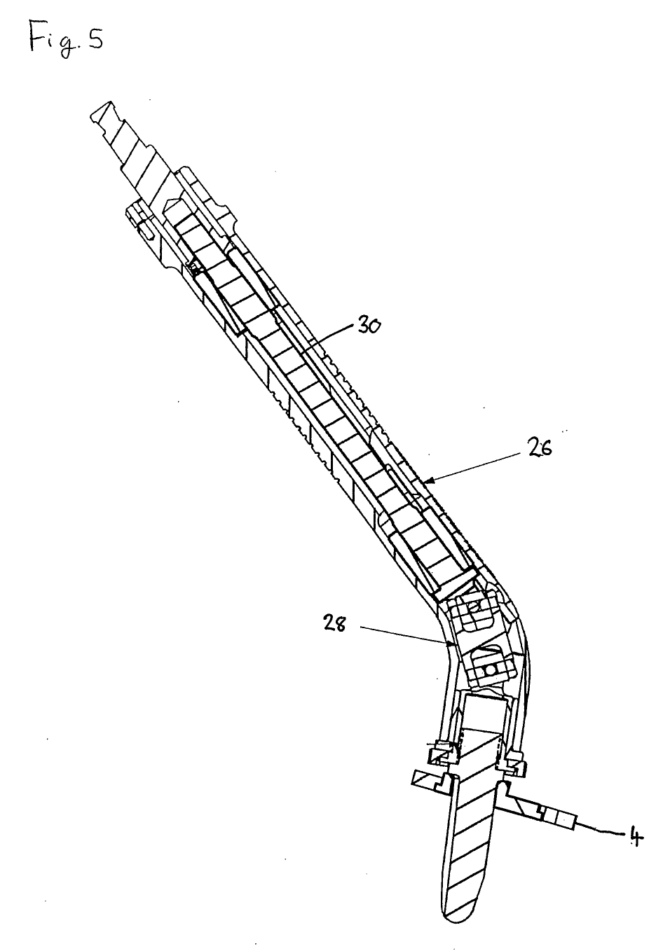

FIG. 5 is a cross section of the assembly shown in FIG. 4.

A reamer guide 2 according to an embodiment of the present invention is shown in plan view in FIG. 1. The reamer guide 2 is made up of a plate 4 which is of a similar size to the cross section of a proximal tibia and a disc 6 which is rotatably mounted in a circular aperture in the plate 4. As can be seen, the aperture is not wholly enclosed by the plate but has a periphery corresponding to a part of the circumference of a circle. The disc 6 can rotate about its centre which corresponds to an axis perpendicular to plane of the reamer guide 2. A bushing 8 is formed in the disc 6, and has a central longitudinal axis offset from the central axis of rotation of the disc. The reamer guide 2 is manufactured from stainless steel, or another medical grade material, or any other suitable bio-compatible material of sufficient strength.

A cross-section of the reamer guide 2 along line AA′ of FIG. 1 is depicted in FIG. 2. The reamer guide is shown in its operative position located on the resected surface of a tibia 10. A reamer 12 is mounted in the bushing 8 to ream a cavity 14 in the tibia 10. The cavity can be reamed to extend substantially along a longitudinal axis of the tibia. The bushing has a bushing axis which extends along the central longitudinal axis of the circular cross section bushing and is directed toward the axis of rotation of the disc so as to form an acute angle there between.

The disc 6 is retained within the circular aperture by a flange 16 formed on the disc and extending around its periphery which interfaces with a corresponding lip or formation 18 formed in the edge of the circular aperture. The lip 18 and flange 16 ensure that the disc cannot be removed from the plate in the direction away from the exposed surface of the guide in use. The resected surface of the tibia 10 prevents the disc 6 from being removed in the direction towards the tibia 10. This construction allows the assembly of the plate 4 and disc 6 to be secure in use and yet also easily disassembled for cleaning.

The plate 4 incorporates first and second holes 20 disposed in plate 4 on opposed sides of the aperture and through which pins (not shown), or other fixings, can be inserted to secure the plate 4 on the resected surface of the tibia 10.

The bushing 8 includes a shoulder which raises above the surface of the disc 6. This shoulder can engage with a stop 22 on the shaft of the reamer to limit the travel of the reamer along the bushing axis so as to prevent the reamer accidentally reaming too deeply into the tibia 10. In this embodiment the projection 22 is fixed in position on the reamer 12, although in other embodiments, the position of the stop or of the shoulder can be adjustable to allow the reaming depth to be altered. The combination of the reaming depth and angle of inclination of the reaming axis determine the shape of the cavity formed.

As mentioned above, it can be seen that the longitudinal axis of the aperture, or reaming bore, defined by the bushing 8 is angled at an acute angle to the axis of the central axis 24 of the disc 6. Thus, as the reamer 12 is inserted further into the bushing 8, the radial distance from its tip to the central axis 24 reduces.

FIG. 3 shows an isometric view of an assembled reamer guide and reamer (the tibia is not shown). A distal end of reamer 12 has a screw thread about an outer surface providing a connector for engaging a drive mechanism. Other connectors can be used to connect the reamer to a drive. FIG. 4 shows a drive system attached to the assembly of FIG. 3. A cross section through this assembly is shown in FIG. 5. The drive mechanism is contained in a casing 26. This casing 26 contains a double universal joint 28. One end of the double universal joint is attached to a driven end of the reamer 12 by a screw thread. The other end of the double universal joint 28 is attached to a drive shaft 30. At its free end, the drive shaft 30 includes a connector 32 for receiving a rotational drive.

The use of the double universal joint 28 allows the reamer to be rotatably driven with no requirement for the rotational drive to be coaxial with the reamer axis. This facilitates the use of the assembly with a small incision and resulting small working space available with a minimally invasive surgical procedure. The universal joint 28 co-operates with the reamer to supply drive to the reamer and a constant angle between the longitudinal axis of the reamer 12 and the longitudinal axis of the casing 26 is maintained as the rotatable disc 6 is rotated within the plate 4.

In an alternative embodiment of the invention, the bushing is parallel to the axis of rotation of the plate, allowing a cylindrical cavity to be reamed.

In another alternative embodiment of the invention, the universal joint can be substituted with a gear arrangement, a hydraulic drive or a pneumatic drive to allow the reamer to be driven by a drive which is not coaxial with the reamer.

In a further alternative embodiment of the invention, the projection on the reamer can be adjusted in position to allow reaming to various depths instead of a single fixed depth.

By controlling the depth of reaming and/or the reaming angle a range of different cavity shapes can be created extending from a circular cylindrical cavity, through frusto conical cavities to conical cavities.

A surgical procedure utilising the above described reamer guide assembly will now be described. As will be appreciated various surgical operations are carried out before and after the operations described below, but these have not been described so as not to obscure the nature of the present invention. The surgeon resects the proximal surface of the tibia. The reamer guide 2 is then secured on the resected surface using pins such that the centre of the disc 6 is located at the centre of the location where it is desired to form the cavity.

Once the reamer guide is secured in position, the surgeon can then introduce a reamer into the bushing 8 and attach it to a drive mechanism assembly housing in casing 26. The reamer is then rotated to ream a cavity into the tibia. After reaming to a specific depth, the disc 6 is rotated so as to increase the size of the cavity and eventually the disc is rotated through 360 degrees so as to form an inverted conical shape cavity in the tibia. A cavity larger than the reamer 12 is therefore formed in the tibia. The cavity has an overall conical section depending on the angle of the bushing 8 and depth of ream.

In a preferred embodiment of the method, the surgeon reams down to the full depth of the desired cavity before rotating the disc. In this way, the described reamer drive can pass the femur without full subluxation

In another embodiment of the method, the surgeon rotates the disc before the full depth of cavity has been reamed. This embodiment will require more than one rotation of the disc to ream the complete cavity.

Although the reaming guide, assembly and method have been described in relation to creating a cavity in a tibia, it will be appreciated that they can also be used to form cavities in other bones, and are particularly useful in minimally invasive surgery techniques using a restricted size surgical site.

1. A reamer guide for reaming a cavity within a tibia, the reamer guide comprising:
a plate having a circular aperture there through and defining a plane; and
a disc rotatably mounted in the aperture, the disc having a bushing therein defining a reamer axis offset from the centre of the disc for receiving a reamer in use and in which the disc is rotatable about an axis transverse to the plane of the plate.

2. A reamer guide as claimed in claim 1, wherein the plate further includes at least one mounting hole.

3. A reamer guide as claimed in claim 1 or 2, in which the plate further comprising an inner circular edge having a first formation therein, and wherein the periphery of the disc has a matching formation which engages with the first formation to retain the disc within the aperture.

4. A reamer guide as claimed in claim 3, in which the circular formation is a shoulder and the matching formation is a flange.

5. A reamer guide as claimed in any one of the preceding claims, wherein the reamer axis is angled toward the axis of rotation of the disc.

6. A reamer guide as claimed in any preceding claim, the bushing including a stop toward a free end for limiting the travel of a reamer into the bushing.

7. An assembly including:
a reamer guide according to any of claims 1 to 6; and
a reamer sized to substantially match the inner diameter of the busing.

8. An assembly as claimed in claim 7, wherein the reamer further includes a projection sized to engage with an edge of a free end of the bushing, the projection positioned a distance from a distal end of the reamer to control the depth of the cavity to be formed to correspond to a desired depth.

9. An assembly as claimed in claim 7 or 8, further comprising a universal joint attached to a proximal end of the reamer for transmitting torque to the reamer about the axis between the distal and proximal ends.

10. An assembly as claimed in any one of claims 7 to 9, further comprising a drive mechanism attached to the universal joint for rotating the reamer.

11. A method of reaming a cavity within a tibia, the method comprising:
resecting a surface of the tibia in which the cavity is to be reamed;
locating a reamer guide on the resected surface, the reamer guide having a rotating disc with a bushing offset from the centre of rotation of the disc, such that the centre of the disc is located above the desired centre of the cavity;
attaching a drive mechanism to a reamer, the drive mechanism extending at least partially at an acute angle to the longitudinal axis of the reamer;
reaming the tibia through the bushing with the reamer; and
rotating the disc while still driving the reamer, thereby enlarging the cavity.

12. The method of claim 11, wherein reaming through the bushing includes reaming to a predetermined depth before rotating the disc.

13. A method as claimed in claim 11 or 12, further comprising securing the reamer guide on the resected surface before beginning reaming.

14. A reamer guide substantially as hereinbefore described with reference to the accompanying drawings.

15. An assembly of a reamer guide and a reamer substantially as hereinbefore described with reference to the accompanying drawings.
